(19) United States
(12) Patent Application Publication
(10) Pub. No.: US 2023/0082493 A1
(43) Pub. Date: Mar. 16, 2023

(54) NEEDLE DEVICES FOR ACCESSING LYMPH NODES AND THORACIC DUCTS

(71) Applicant: SUREAX, INC., San Diego, CA (US)

(72) Inventor: Ghassan S. Kassab, La Jolla, CA (US)

(73) Assignee: SUREAX, INC., San Diego, CA (US)

(21) Appl. No.: 17/794,849

(22) PCT Filed: Jan. 25, 2021
(86) PCT No.: PCT/US2021/014971

§ 371 Date: Jul. 22, 2022

Related U.S. Application Data
(60) Provisional application No. 62/965,154, filed on Jan. 23, 2020.
(60) Provisional application No. 62/965,159, filed on Jan. 23, 2020.

(51) Int. Cl. A61B 17/34

(52) U.S. Cl. CPC A61B 17/3478; A61B 2017/3488

(57) ABSTRACT

Needle devices for accessing lymph nodes and thoracic ducts. A needle device for insertion into a lymph node and secured after access to the lymph node is described. The needle can aspirate or introduce fluids through a needle aperture in the needle sidewall. The needle may be curved. The needle device may also have a channel for fluids. The channel can have an aperture in the sidewall or be a separate channel alongside the lumen and have a distal aperture. Securing the needle can be accomplished via a hook, spiraling element, balloon or glue, which can be introduced via an open needle tip or a channel aperture. The spiraling element may be attached to a drive configured to rotate the spiraling element.

### PRIORITY

The present patent application is related to, and claims the priority benefit of, U.S. Provisional Patent Application Ser. No. 62/965,154, filed on Jan. 23, 2020, and is also related to, and claims the priority benefit of, U.S. Provisional Patent Application Ser. No. 62/965,159 filed on Jan. 23, 2020, the contents of which are hereby incorporated by reference in their entirety into this disclosure.

### BACKGROUND

The lymphatic system is a part of the circulatory system and the immune system. It is comprised of a large network of lymphatic vessels and lymphatic organs that carry lymph through the body. Lymph contains lymphocytes and also waste products and cellular debris together with bacteria and proteins. Lymphocytes are concentrated in the lymph nodes.

For diagnosing certain diseases, lymphangiography can be useful to image the lymphatic system (LS). For example, lymphangiography can be used to detect chylous leakage thereby indicating where surgical intervention may be required. Traditional pedal lymphangiography; i.e., direct cannulation of the lymphatic vessels, has significant technical, time cost and image quality challenges.

Recently, the field of lymphatic imaging and intervention, underwent revival due to development of intranodal lymphangiography (IL) and thoracic duct embolization (TDE). IL was developed as a technically simple alternative to pedal lymphangiography. Intranodal lymphangiography is faster and less invasive than the standard pedal lymphangiography. IL is performed by accessing the lymph nodes with a spinal needle using ultrasound guidance (USG). It also provides superior to PL imaging and now is the leading method of imaging of the lymphatic system worldwide. Although the IL technique has many advantages over PL, there are remaining problems because the needle used is borrowed from spinal procedures and is not designed for lymphangiography. These limitations include: 1) Difficulty of introducing needle into LN, because of mobility of the LN positioned in loose subcutaneous tissue; 2) Difficulty in positioning of the needle precisely in the central part of the LN; 3) Lack of stabilization mechanism of the needle in LN during intervention and patient transfer. The last issue results in frequent dislodgement of the needle, greatly impairing the quality of the imaging and success of the lymphatic procedures.

Thoracic duct embolization is a minimally invasive technique developed to treat the pulmonary lymphatic disorders. During thoracic duct embolization, the access to the thoracic duct (TD) is obtained transabdominally using a long 21-22 G needle. Similarly, the needle is not specially designed for TD intervention with the following shortcomings: 1) The needle are not sharp enough to traverse long route of complain tissue; 2) It is not sufficiency rigid, so it is difficult to perform fine control of the tip of the needle; 3) The sharp angle of the access to the TD makes it difficult to manipulate the wire in the TD, that is frequently sheared; 4) The choice of needle length is very limited. Lymphatic imaging and TD interventions are both time-consuming and technically challenging and remain a major hurdle to the detection and treatment of lymphatic disorders.

Therefore, innovative lymphatic system access tools are critical to the progress of lymphatic intervention. However, there is a lack of dedicated lymphatic intervention equipment which presents technical challenges in performing the lymphatic procedures and hence limits full adoption.

Solving this problem allows for the wider adoption of intranodal lymphangiography thereby providing patients with faster and less invasive diagnosis of their disease. Therefore there is a need in the art for needles that can be secured within the lymph nodes for the purposes of intranodal lymphangiography

### BRIEF SUMMARY

The present disclosure includes disclosure of a needle device, comprising a needle having a needle lumen defined therethrough terminating at a needle aperture at or near a distal end of the needle, the needle device configured to reversibly engage a tissue and further configured to introduce a fluid through the needle lumen into the tissue or a lumen thereof. The present disclosure includes disclosure of a needle device, further comprising a securing device positioned within the needle lumen, the spiraling element configured to at least partially protrude from the needle aperture and to reversibly engage a tissue. The present disclosure includes disclosure of a needle device, wherein the securing device comprises a spiraling element, and wherein the spiraling element is configured to reversibly engage a lymph node.

The present disclosure includes disclosure of a needle device, further comprising a drive assembly configured to protrude the securing device from the needle aperture and to retract the securing device into the needle aperture. The present disclosure includes disclosure of a needle device, wherein the drive assembly comprises a spring-loaded mechanism operably connected to the spiraling element to propel the spiraling element into the lymph node. The present disclosure includes disclosure of a needle device, wherein the spring-loaded mechanism is configured to be manually triggered using a manual mechanism. The present disclosure includes disclosure of a needle device, wherein the spiraling element comprises a helical distal end terminating with a sharp and straight distal tip.

The present disclosure includes disclosure of a needle device, wherein the needle has a distal portion bent at an angle relative to a longitudinal axis of a longitudinal portion of the needle. The present disclosure includes disclosure of a needle device, wherein the angle is at or between 14 degrees and 20 degrees. The present disclosure includes disclosure of a needle device, wherein the distal portion is at or about 5 mm long. The present disclosure includes disclosure of a needle device, configured for guidewire exchange using a guidewire having a diameter of 0.014″.

The present disclosure includes disclosure of a needle device, wherein the distal end of the needle is beveled. The present disclosure includes disclosure of a needle device, wherein the bevel is at or about 15 degrees relative to the longitudinal axis. The present disclosure includes disclosure of a needle device, wherein the distal end of the needle is configured to puncture a thoracic duct.

The present disclosure includes disclosure of a needle device, forming part of a kit, the kit further comprising a second needle device, and a guidewire. The present disclosure includes disclosure of a kit, wherein the needle device is configured to reversibly engage a lymph node, and wherein the second needle device is configured to puncture a thoracic duct. The present disclosure includes disclosure of a kit, further comprising a microcatheter configured to cannulate the thoracic duct over the guidewire.

The present disclosure includes disclosure of a needle device, wherein the needle aperture is positioned through a sidewall of the needle proximal to the distal end of the needle. The present disclosure includes disclosure of a needle device, further comprising a channel running parallel to the needle, the channel comprising a channel lumen and a channel aperture, wherein the channel aperture and the channel lumen are in fluid communication, the channel lumen disposed within the needle lumen, and the channel aperture is disposed in the needle sidewall proximal of the needle aperture. The present disclosure includes disclosure of a needle device, further comprising a securing means disposed in the channel lumen.

The present disclosure includes disclosure of a needle device, wherein the distal end comprises a curved distal tip. The present disclosure includes disclosure of a needle device, wherein the distal tip has a closed end. The present disclosure includes disclosure of a needle device, wherein the channel lumen and the needle lumen are not in fluid communication with each other. The present disclosure includes disclosure of a needle device, wherein the securing means is selected from the group consisting of a hook, a spiraling element, and glue. The present disclosure includes disclosure of a needle device, wherein the securing means is a spiraling element, and wherein the needle device further comprises a drive assembly attached to the spiraling element and configured to rotate the spiraling element.

The present disclosure includes disclosure of a needle device, comprising a needle having a needle lumen defined therethrough terminating at a needle aperture at or near a distal end of the needle, and a securing device positioned within the needle lumen, the spiraling element configured to at least partially protrude from the needle aperture and to reversibly engage a tissue, the needle device configured to reversibly puncture a tissue and further configured to introduce a fluid through the needle lumen into the tissue or a lumen thereof. The present disclosure includes disclosure of a needle device, wherein the securing device comprises a spiraling element, and wherein the spiraling element is configured to reversibly engage a lymph node.

The present disclosure includes disclosure of a needle device, further comprising a drive assembly configured to protrude the securing device from the needle aperture and to retract the securing device into the needle aperture. The present disclosure includes disclosure of a needle device, wherein the drive assembly comprises a spring-loaded mechanism operably connected to the spiraling element to propel the spiraling element into the lymph node. The present disclosure includes disclosure of a needle device, wherein the spring-loaded mechanism is configured to be manually triggered using a manual mechanism.

The present disclosure includes disclosure of a needle device, wherein the spiraling element comprises a helical distal end terminating with a sharp and straight distal tip. The present disclosure includes disclosure of a needle device, wherein the needle has a distal portion bent at an angle relative to a longitudinal axis of a longitudinal portion of the needle. The present disclosure includes disclosure of a needle device, wherein the angle is at or between 14 degrees and 20 degrees. The present disclosure includes disclosure of a needle device, wherein the distal portion is at or about 5 mm long.

The present disclosure includes disclosure of a needle device, configured for guidewire exchange using a guidewire having a diameter of 0.014″. The present disclosure includes disclosure of a needle device, wherein the distal end of the needle is beveled. The present disclosure includes disclosure of a needle device, wherein the bevel is at or about 15 degrees relative to the longitudinal axis. The present disclosure includes disclosure of a needle device, wherein the distal end of the needle is configured to puncture a thoracic duct.

The present disclosure includes disclosure of a needle device, forming part of a kit, the kit further comprising a second needle device, and a guidewire. The present disclosure includes disclosure of a kit, wherein the needle device is configured to reversibly engage a lymph node, and wherein the second needle device is configured to puncture a thoracic duct. The present disclosure includes disclosure of a kit, further comprising a microcatheter configured to cannulate the thoracic duct over the guidewire.

The present disclosure includes disclosure of a needle device for accessing a lymph node and securing the needle in place, the device comprising a needle having a distal tip, a needle lumen, a sidewall, a needle aperture in the sidewall proximal of the distal tip, wherein the needle lumen and the needle aperture are in fluid communication, and a channel running parallel to the needle, the channel comprising a channel lumen and a channel aperture, wherein the channel aperture and the channel lumen are in fluid communication, the channel lumen disposed within the needle lumen, and the channel aperture is disposed in the needle sidewall proximal of the needle aperture, and a securing means disposed in the channel lumen. The present disclosure includes disclosure of a needle device, wherein the distal tip is curved. The present disclosure includes disclosure of a needle device, further comprising a closed end at the distal tip.

The present disclosure includes disclosure of a needle device, wherein the channel lumen and the needle lumen are not in fluid communication with each other. The present disclosure includes disclosure of a needle device, wherein the securing means is selected from the group consisting of a hook, a spiraling element, and glue. The present disclosure includes disclosure of a needle device, wherein the securing means is a spiraling element, and the device further comprises a drive assembly attached to the spiraling element and configured to rotate the spiraling element.

The present disclosure includes disclosure of a needle device for accessing a lymph node and securing the needle in place, the device comprising a needle having a distal tip, a needle lumen, a sidewall, a needle aperture in the sidewall proximal of the distal tip, wherein the needle lumen and the needle aperture are in fluid communication, and a channel running parallel to the needle, the channel comprising a channel lumen and a channel aperture, wherein the channel aperture and the channel lumen are in fluid communication, wherein the channel lumen is disposed outside the needle lumen the channel aperture is disposed outside the needle lumen, and a securing means disposed in the channel lumen. The present disclosure includes disclosure of a needle device, where the distal tip is curved. The present disclosure includes disclosure of a needle device, further comprising a closed end at the distal tip.

The present disclosure includes disclosure of a needle device, wherein the channel lumen and the needle lumen are not in fluid communication with each other. The present disclosure includes disclosure of a needle device, wherein the securing means is one of a hook, a spiraling element, or glue. The present disclosure includes disclosure of a needle device, wherein the securing means is a spiraling element and the device further comprises a drive assembly, wherein the drive assembly is attached to the spiraling element and activation of the drive assembly rotates the spiraling element.

The present disclosure includes disclosure of a method for using a needle device to access a lymph node the method comprising the steps of inserting a needle into the lymph node of a patient, wherein the needle comprises a sidewall and a needle aperture, deploying a securing means to attach the needle device to the lymph node; and introducing contrast media into the lymph node. The present disclosure includes disclosure of a method, wherein the needle also comprises a channel and a channel aperture; and the step of deploying a securing means further comprises the step of deploying the securing means through the channel aperture. The present disclosure includes disclosure of a method, wherein the step of introducing contrast media comprises the step of introducing contrast lumen through the needle aperture.

The present disclosure includes disclosure of a method, wherein the step of inserting a needle into the lymph node further comprises the step of, positioning the channel aperture outside of the lymph node. The present disclosure includes disclosure of a method, wherein step of deploying a securing means comprises the step of deploying the securing means through a channel aperture positioned outside the needle lumen. The present disclosure includes disclosure of a method, wherein the step of deploying a securing means further comprises the step of deploying the securing means through the sidewall. The present disclosure includes disclosure of a method, further comprising the method of rotating the securing means to secure the needle to the lymph node.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIGS. 1, 2, 3, and 4 show needle devices accessing lymph nodes, according to exemplary embodiments of the present disclosure;

FIG. 5 shows a curved needle and hub for accessing a lymph node, according to an exemplary embodiment of the present disclosure

FIG. 6 shows a straight needle and needle tip detail for accessing a lymph node, according to an exemplary embodiment of the present disclosure

FIG. 7 shows a drive assembly to rotate a securing device, according to an exemplary embodiment of the present disclosure;

FIG. 8 shows a securing device, according to an exemplary embodiment of the present disclosure;

FIG. 9 shows a needle accessing a lymph node, according to an exemplary embodiment of the present disclosure;

FIG. 10 shows a needle device configured to engage a lymph node, according to an exemplary embodiment of the present disclosure;

FIG. 11 shows a needle device configured to puncture a thoracic duct, according to an exemplary embodiment of the present disclosure; and

FIG. 12 shows a block diagram of components of a kit, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “distal” is used to refer to the part of the device farthest from an operator or closest to a patient. The term “proximal” is used to describe the part of the device closest to an operator or farthest from a patient. For example, the part of the needle that is inserted into the patient first will be the distal part.

### Needle Devices

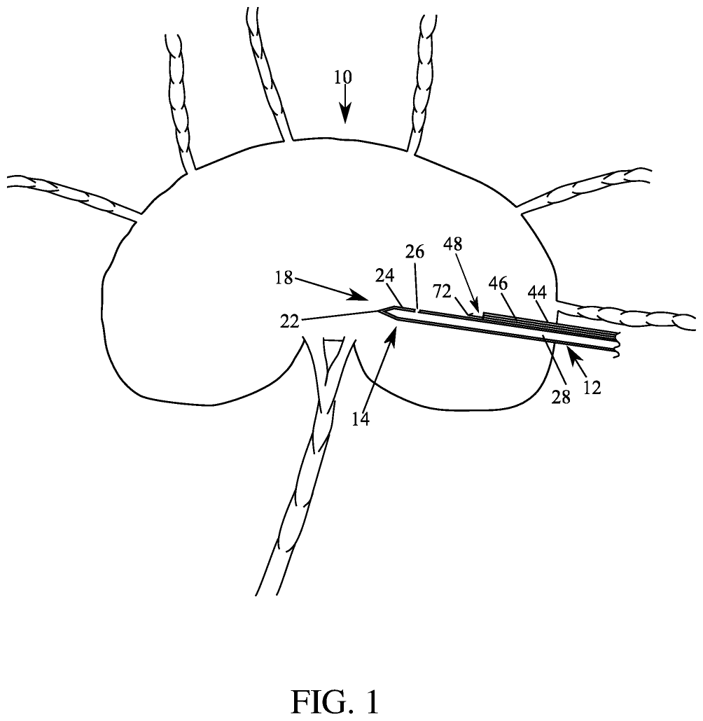

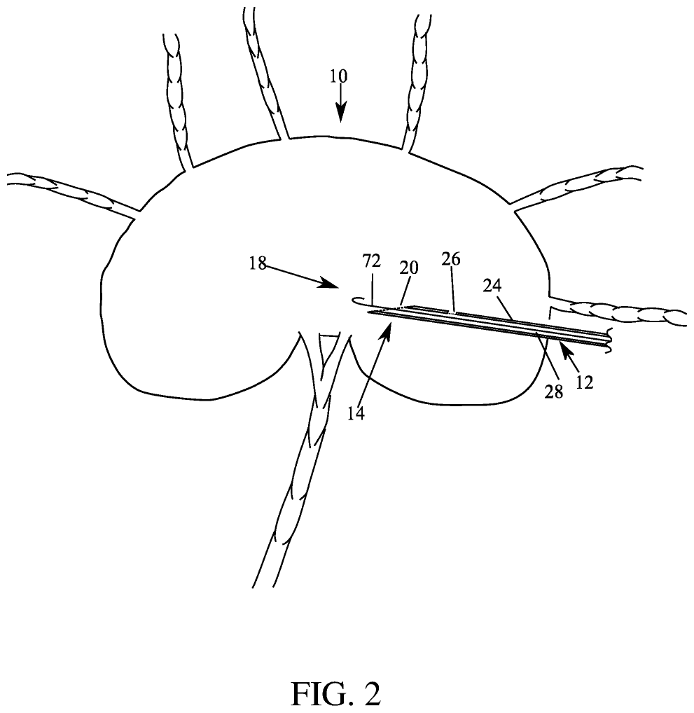

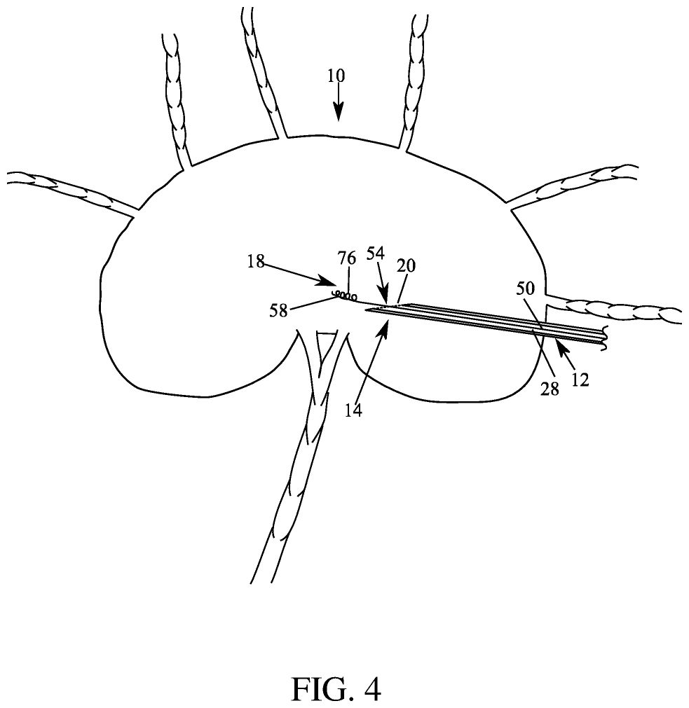

In one aspect of the invention, various embodiments of the invention comprise a needle 12 as shown in FIGS. 1-4. The needle 12 comprises a distal end 14 comprising a distal tip 18 and a sidewall 24. The distal tip 18 is sharp to aid in puncturing tissue and comprises a closed tip 22. Some embodiments, such as shown in FIGS. 2 and 4 comprise an open tip needle.

In the embodiments of FIGS. 1-3, a needle aperture 26 is disposed in the sidewall 24 near the distal tip 18. The needle aperture 26 allows for fluid communication between a needle lumen 28 and the exterior of the needle 12. For example, contrast media can be introduced via a syringe or other method into a needle lumen 28, and then travel through the needle lumen 28 to exit the needle aperture 26. The needle lumen 28 may end at the needle aperture 26 or extend to the distal tip 18. The needle aperture 26 may be positioned 1-100 mm away from the distal tip 18 of the needle 12. In other embodiments, the needle aperture 26 can be 1-50 mm, 1-25 mm, 1-10 mm, 1-5 mm, or 1-2 mm away from the distal tip 18.

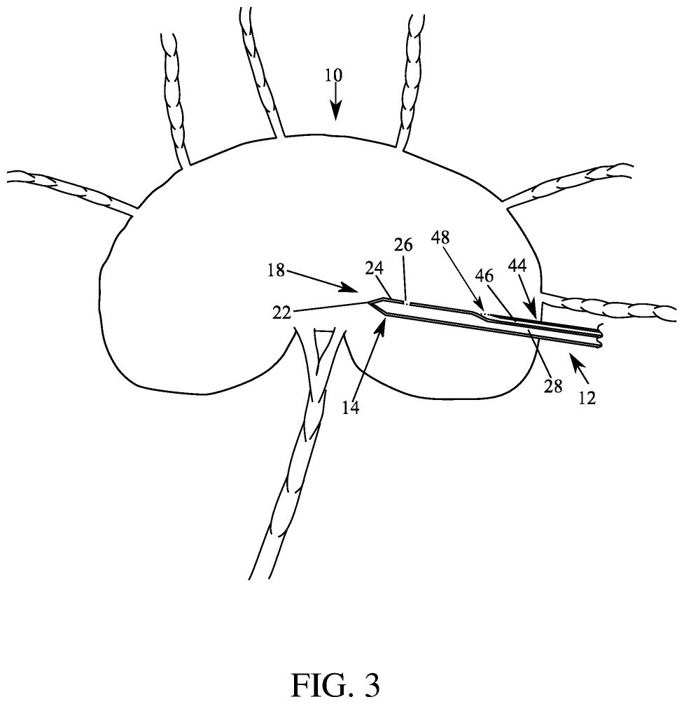

In another embodiment of the present invention, the needle 12 comprises a separate channel 44 as shown in FIGS. 1 and 3. The channel 44 may comprise a channel lumen 46 isolated from the needle lumen 28. That is, the two lumens are not in communication along their length. The channel 44 and channel lumen 46 extend parallel to the needle lumen 28. The channel 44 also comprises a channel aperture 48 at the distal end 14 of the needle, and proximal of the needle aperture 26. Similar to the needle aperture 26, the channel aperture 48 is located near the distal tip 18 of the needle 12 and allows communication between a channel lumen 46 and the exterior of the needle 12. In the embodiment, the channel lumen ends at the channel aperture 48.

In one embodiment of the channel as shown in FIG. 3, the channel 44 and channel lumen 46 are positioned within the needle 12, and the channel aperture 48 is in the sidewall of the needle. In the embodiment of FIG. 1, the channel is positioned exterior to the needle and the channel aperture is exterior to the needle sidewall.

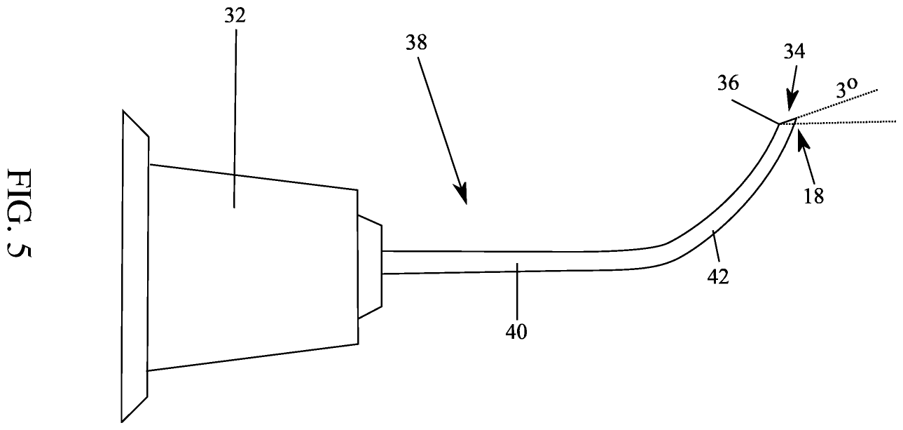

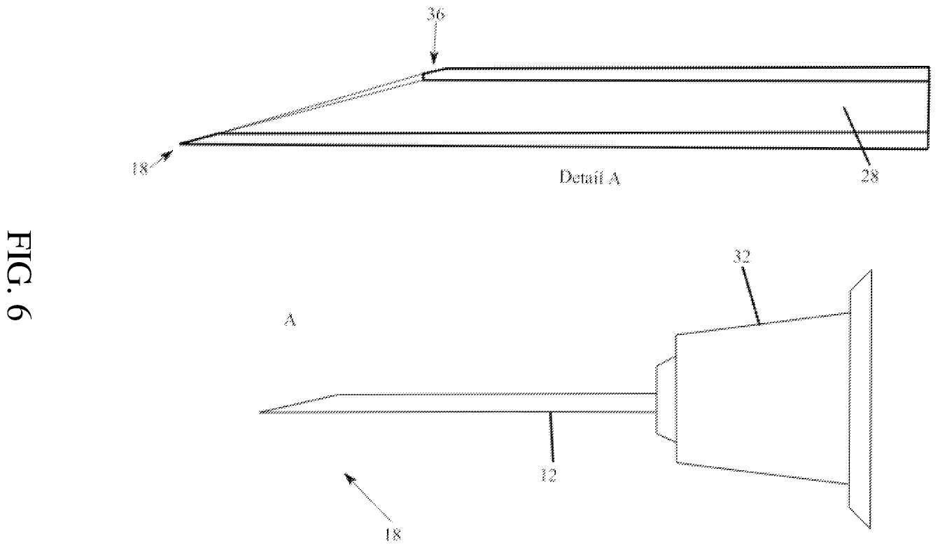

Another embodiment of the present invention comprises a needle 38 having a curved distal portion 42. As shown in FIG. 5, the curved needle 38 may comprise a proximal straight portion 40 which extends from a hub 32 along a longitudinal axis much like a straight needle. The curved needle 38 further comprises a curved portion 42 distal of the initial straight portion 40. The curved portion 42 veers from the longitudinal axis in a lateral direction for a distance of about 0.06-0.08 inches. In a preferred embodiment, the free length of the curved needle 38 is about 9.9 inches and the diameter of the needle is 0.32 inches. The curved needle 38 distal tip 18 is also beveled 34. As the needle 38 is curved, the bevel 34 will form an angle approximately parallel to the longitudinal axis. The angle of the bevel 34 can vary up to 3 degrees. The curved needle 38 will also be anti-coring, and comprise anti-coring features similar to the straight needle 12, as previously described such as the bevel.

In one embodiment, the present invention comprises a straight needle 12 as in FIG. 6. The needle 12 extends along a longitudinal axis. In this embodiment, the needle 12 protrudes from a hub 32 and has a free length of about 5.7 inches, wherein the free length is measured from the distal tip 18 of the needle 12 to the point where the needle 12 protrudes from a hub 32. The needle 12 preferably has a diameter of about 0.028 inches. The distal tip 18 of the needle 12 is open 20 and comprises another bevel 34. The bevel 34 preferably forms an angle of about 15 degrees relative to the longitudinal axis. The needle 12 is an anti-coring needle. To facilitate the anti-coring function, the heel 36, or proximal part of the bevel 34, may comprise a break edge, be blasted to dull the heel, or otherwise be dulled.

The described needles 12, 38 may share features, such as the sidewall 24, needle aperture 26, channel 44 and channel aperture 48 as described. For example, although FIGS. 1-4 depict a straight portion of a needle, it is understood that the needle portion depicted could be the curved portion of the curved needle.

### Securing Devices/Means

In another embodiment, a securing device 50 is slidingly disposed within the channel lumen 46. The securing device 50 may be a deployable hook 72, glue 74, spiraling element 76, balloon or other as known in the art. The deployable hook 72 can have any shape that deploys into surrounding tissue and secures the needle 12. Shapes may be, and are not limited to: an arrow, curved, hook, or any other.

In the embodiment shown in FIG. 1, the deployable hook 72 is disposed in the needle lumen 28 of a needle without a channel 44. In this embodiment, the needle 12 comprises an open tip 20. The deployable hook 72 can extend through the open tip 20. When the hook 72 is deployed through the open tip 20, the hook 72 obstructs the open tip 20 such that no liquid can pass through the open tip 20 and contrast media must exit the needle aperture 26 in the sidewall 24.

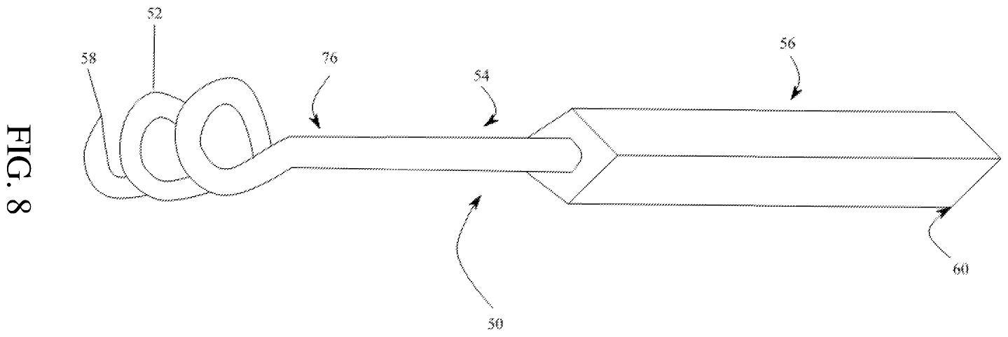

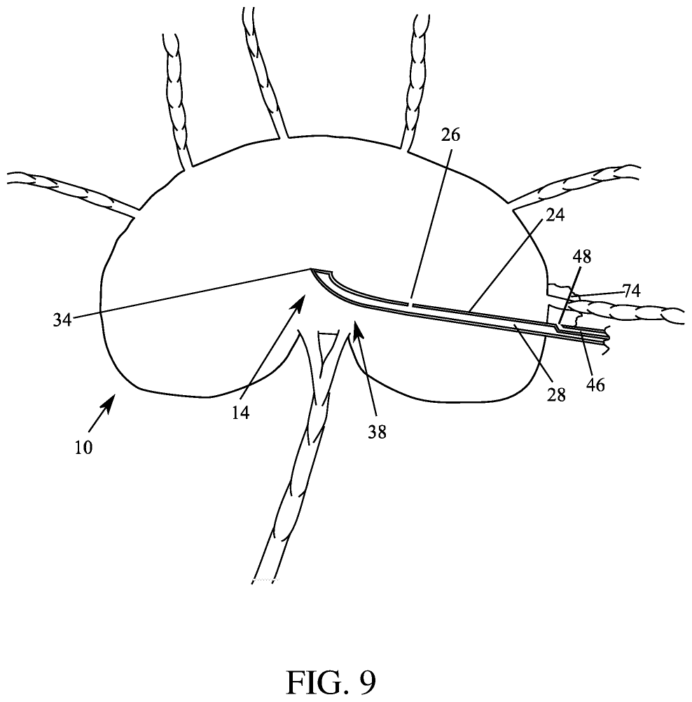

In another embodiment, as shown in FIG. 9, the securing device 50 may comprise glue or other bioadhesive compound 74. In this embodiment, the glue 74 is housed within a needle 12 having a closed tip 22 and the channel aperture 48 is disposed sufficiently proximal the needle aperture 26 such that the glue 74 will not travel to the needle aperture or lymph node 10 when deployed. The apertures may be positioned so that when the needle is inserted into the lymph node, the needle aperture is within the lymph node and the channel aperture is exterior to the lymph node. Thus glue will adhere the needle to the exterior surface or surrounding tissue of the lymph node and contrast media will be separate and introduced within the lymph node. Furthermore, as the needle lumen 28 and channel lumen 46 are isolated, the contrast media and glue 74 do not mix within the needle 12.

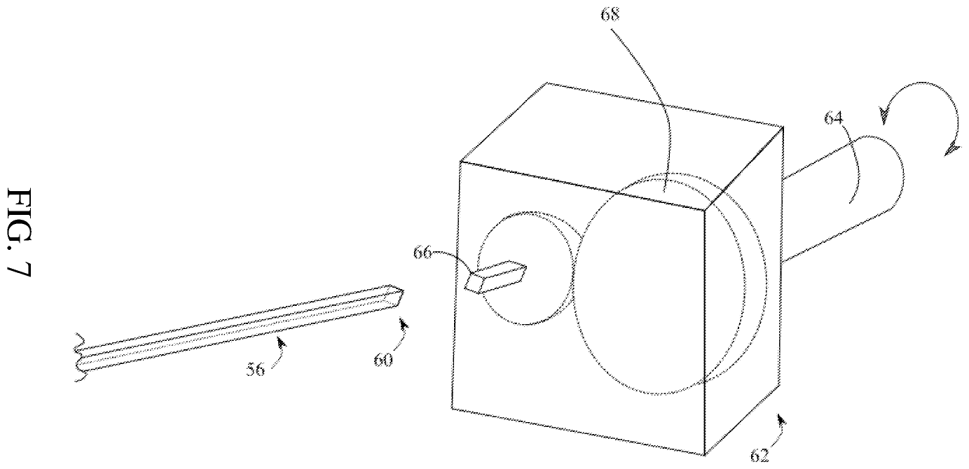

In another aspect of the invention, the securing device 50 comprises a spiraling element 76 as in FIG. 8. The spiraling element 76 comprises a proximal end 56. The proximal end 56 can comprise a square profile at its proximal tip 60 to mate to a drive sleeve 66 of a drive assembly 62. In a preferred embodiment, the proximal end 56 is made of 22 gauge thin wall hypo tubing.

The spiraling element 76 also comprises a distal end 54. The distal end 54 of the spiraling element 76 comprises a sharpened distal tip 58, preferably formed from a close wound flat wire coil. The distal tip 58 comprises a spiral/coil 52 of approximately 0.5 inches in two places having a pitch of 0.03 to 0.04 inches. In a preferred embodiment, the wire is a 0.003 by 0.006 inch flat wire, and has a 0.012 inch inner diameter and a 0.018 outer diameter. In another embodiment, deploying the spiraling element 76 extends the distal tip 58 of the spiraling element 76 out of the needle tip 18 for approximately 6-8 millimeters.

In a further embodiment, a proximal portion of the spiraling element's distal end 54 and a distal portion of the spiraling element's proximal end 56 are bonded together along their length creating an area of overlap for 0.5 inches.

In use, the sharp distal tip 58 of the spiraling element 76 is introduced into tissue 10 and then rotated so that the spiral 52 of the spiraling element 76 is advanced deeper into the tissue 10. The spiraling element 76 rotation can be powered by a drive assembly 62. The drive assembly 62 comprises a drive sleeve 66 which attaches to the proximal end 56 of the spiraling element 76. Where the proximal end 56 of the spiraling element 76 comprises a square profile, the drive sleeve 66 comprises a complimentary square profile so that the spiraling element proximal end 56 and the drive sleeve 66 will fit securely together. The drive sleeve 66 extends distally from a housing 70 of the drive assembly 62. The drive assembly 62 also comprises a drive pin 64 which extends proximally from the housing 70. The housing 70 comprises a gearing 68, wherein the gearing 68 is configured such that one turn of the drive pin 64 produces three turns of the spiraling element 76.

In one embodiment the spiraling element 76 is slidingly disposed within the needle lumen 28 and extendable through the open tip 20 of the needle 12. When the spiraling element 76 is extended beyond the open tip 20, contrast media can be pushed past the extended spiraling element 76 as it does not block the open tip 20, such as in FIG. 4.

In another embodiment, the spiraling element 76 is slidingly disposed within the channel lumen 46.

As described above, and as apparent to one of ordinary skill in the art, the constituent elements can be interchanged to produce a device for particular use in accessing and securing to lymph nodes. Some of the interchangeability is discussed further below.

For example, FIG. 1 shows a needle device having a pointed distal tip that is a closed tip. A needle aperture is disposed on the sidewall of the needle. The device also comprises a channel parallel to and outside of the needle lumen. Deployed within the channel lumen is a securing means, in this embodiment a hook.

The present disclosure also contemplates, as shown in FIGS. 2-4, any suitable securing means such as glue, a spiraling element, or others known in the art which may be utilized in place of the hook. Additionally, FIG. 2 shows a curved hook, as opposed to an acute hook as in FIG. 1.

The channel is similarly interchangeable and may be disposed on the interior of the needle such that the profile of the needle is preserved, such as in FIG. 3. It follows that any suitable securing means can be used with any positioned channel. In both embodiments of FIGS. 1 and 3, the channel aperture is disposed proximal of the needle aperture.

The present disclosure also contemplates open tip needles, as in FIGS. 2 and 4. As described above, in an open tip needle, said needle is configured to force contrast lumen out the needle aperture, as the securing means would be blocking the open tip. In the case of a closed tip, the contrast would be introduced into the needle lumen. However, it is within the scope of this disclosure that the contrast could be introduced into the channel lumen and the securing means introduced into the needle lumen.

As the closed tip and open tip are interchangeable with previously mentioned features, both tips may have a bevel edge as described above and as shown in FIGS. 5-6. In this embodiment, the bevel provides an anti-coring function to the needle.

Also interchangeable is the shape of the needle. A curved needle, as in FIG. 5-6 can be used with the invention of the present disclosure.

Thus, it is contemplated that any combination of open or closed, curved or straight needle, securing means, and interior or exterior channel could be utilized to access and secure the device to the lymph node.

### Methods of Use

In an embodiment of the present invention, a method of use for a needle for accessing lymph nodes comprises the steps of inserting the needle into the lymph node of a patient; deploying the securing device; and introducing contrast media through the needle.

In an alternate embodiment of a method of use for a needle for accessing lymph nodes, the step of inserting the needle into the lymph node of a patient is guided by imaging such as ultrasound or other appropriate method.

In an alternate embodiment of a method of use for a needle for accessing lymph nodes, the step of deploying the securing device is performed by extending the securing device through the open tip of the needle.

In an alternate embodiment of a method of use for a needle for accessing lymph nodes, the step of deploying the securing device is performed by sliding the securing device through the channel lumen and extending the securing device through the channel aperture.

In an alternate embodiment of a method of use for a needle for accessing lymph nodes, the step of deploying the securing device is performed by sliding the securing device through the needle lumen and extending the securing device through the open tip.

In an alternate embodiment of a method of use for a needle for accessing lymph nodes, the step of introducing contrast media through the needle is performed by introducing the media through the open tip of the needle. In an alternate embodiment of a method of use for a needle for accessing lymph nodes, the step of introducing contrast media through the needle is performed by introducing the media through the needle aperture in the sidewall of the needle.

In an alternate embodiment of a method of use for a needle for accessing lymph nodes, the step of deploying the securing device is performed by extending the securing device through the needle lumen and the open tip, and the step of introducing contrast media is performed by introducing the contrast media through the needle aperture of the sidewall of the needle. In an alternate embodiment of a method of use for a needle for accessing lymph nodes, the step of deploying the securing device is performed by extending the securing device through the channel lumen and channel aperture, and the step of introducing contrast media is performed by introducing the contrast media through the needle aperture of the sidewall of the needle.

In an alternate embodiment of a method of use for a needle for accessing lymph nodes, the step of deploying the securing device is performed by extending the securing device through the open tip and the step of introducing contrast media is performed by introducing the contrast media through the open tip of the needle, where the securing device is a spiraling element.

### Exemplary Embodiments and Methods of Use

1. Lymph Node Access Needle

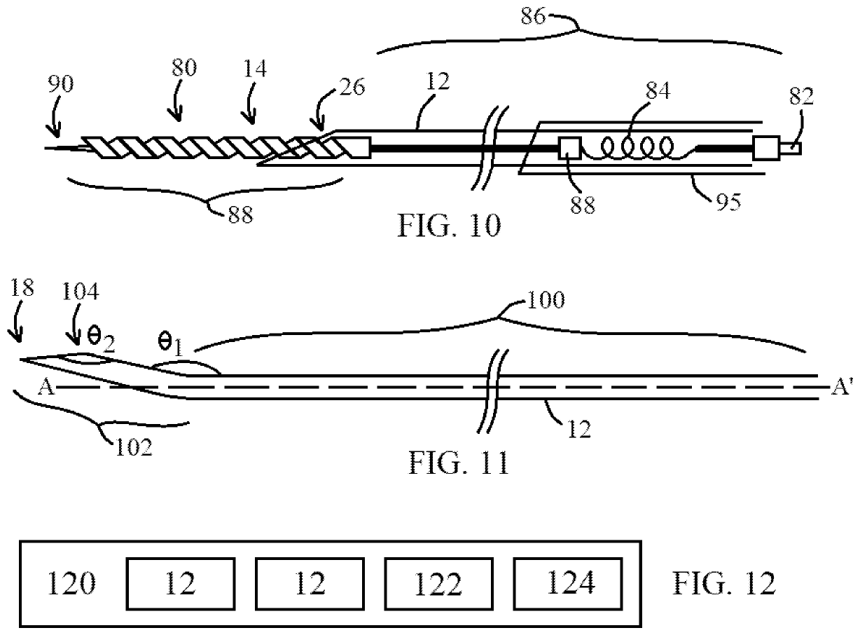

In a preferred embodiment, such as shown in FIG. 10, the present disclosure includes disclosure of an access needle 12, the access needle 12 used to access lymph nodes. An exemplary device comprises a straight needle 12, and a needle aperture 26 at the distal end 14 of the needle 12. Disposed within the needle aperture 26 is a securing device 80, in this case a spiraling element. A manual mechanism 82 (such as a trigger) is connected to and controls a spring-loaded mechanism 84 which is operably connected to the spiraling element 80 to propel the spiraling element 80 into the target lymph node. A drive assembly 86 can be operably connected to the spiraling element 80 to drive the needle 12 into the lymph node.

An exemplary spiraling element 80 is a stylet comprising a helical distal end 88 that extends toward the proximal end of the device. The distal tip 90 of the spiraling element 80 is sharp and straight (along a longitudinal axis of the straight needle 12). The helical structure 80 enables stabilization within the needle 12, thus allowing the tip 90 to pierce the lymph node in a straight manner. As the needle 12 is driven into the lymph node, the helical shape of the spiraling element 80 will also provide stability in the lymph node. The straight and sharp tip 90 will facilitate the puncture instantly by manually triggering a spring 84. At the proximal end, the spiraling element/stylet 80 connects to a drive assembly 86.

The drive assembly comprises a clock spring 84 that is wound to store energy and then locked, such as with a button (an exemplary manual mechanism 82), to retain the stored energy until the spiraling element 80 is deployed. In this embodiment, the proximal end of the spiraling element 80 is a square to connect to a square drive 88 of the drive assembly 86.

In this embodiment, the securing device needle 12 is spring loaded. The spring is triggered manually, preferably by a thumb trigger (an exemplary manual mechanism 82) at the handle of the device. In an exemplary method of use for this embodiment, the spring-loaded mechanism 84 is loaded and the spiraling element 80 is loaded into the needle 12. The clock spring drive is wound to preload the drive assembly 86. The needle 12, with the spiraling element 80 within, is advanced to a point adjacent to the lymph node.

When positioned adjacent to the node, the manual trigger (an exemplary manual mechanism 82) is operated to activate the spring-loaded mechanism 84 which propels the tip 90 of the spiraling element 80 into the lymph node. The clock spring drive mechanism is attached to the square drive of the spiraling element 80 and then activated to unwind the clock spring 84 and drive the helical coil (spiraling element 80) into the node. Where the clock spring drive is locked by a button (an exemplary manual mechanism 82), the button is depressed the clock spring tension is released and the spring 84 unwinds, driving the helical coil (spiraling element 80) into the lymph node. The fibrous nature of the lymph node retains the coil (spiraling element 80) and captures the needle 12 within the node. After the inner stylet is anchored in the lymph node, the outer injection needle 95 will advance to the appropriate position for contrast injection. The space between the stylet and injection needle is adequate for contrast injection. All or several of the components of the LN needle 12 can comprise stainless steel to ensure cost efficiency.

2. Thoracic Duct Access Needle

In another preferred embodiment, the device is a needle access device used to access the thoracic duct. The device comprises a needle 12 having a distal portion 100. The distal portion 100 is bent at an angle relative to a longitudinal portion 102 of the needle 12, such as an angle at or about 18 degrees, or an angle of or about 14 degrees to an angle of or about 20 degrees, for example, relative to the longitudinal axis 104. The bending length (the length of the distal portion 100) that deviates from the straight portion (the longitudinal portion 102) of the needle 12 is at or about 5 mm, for example. The needle 12 is configured to allow for 0.014″ guidewire exchange.

The tip 18 is also sharpened to a bevel 106, the bevel being at or about 15 degrees (for example) as measured from the tip 18 to the longitudinal axis. The bevel slants away from the proximal end of the device, such that the distal end 18 of the bevel is closest to the longitudinal axis when viewed from the side, such as shown in FIG. 11

The present disclosure also includes disclosure of a lymphatic access kit 120, which comprises a lymph node access needle 12 (such as referenced herein) and a thoracic duct access needle 12 (such as referenced herein), and optionally a guidewire 122 (such as a 0.014″ guidewire) and/or a microcatheter 124, such as useful to cannulate the thoracic duct over the guidewire, such as shown in FIG. 12.

While various embodiments of devices for accessing a lymph node and methods for the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A needle device, comprising:
a needle having a needle lumen defined therethrough terminating at a needle aperture at or near a distal end of the needle;
the needle device configured to reversibly engage a tissue and further configured to introduce a fluid through the needle lumen into the tissue or a lumen thereof.

2. (canceled)

3. The needle device of claim 1, further comprising:
a securing device positioned within the needle lumen, the securing device configured to at least partially protrude from the needle aperture and to reversibly engage a tissue, wherein the securing device comprises a spiraling element, and wherein the spiraling element is configured to reversibly engage a lymph node.

4. The needle device of claim 3, further comprising:
a drive assembly configured to protrude the securing device from the needle aperture and to retract the securing device into the needle aperture.

5. The needle device of claim 4, wherein the drive assembly comprises a spring-loaded mechanism operably connected to the spiraling element to propel the spiraling element into the lymph node.

6. (canceled)

7. The needle device of claim 3, wherein the spiraling element comprises a helical distal end terminating with a sharp and straight distal tip.

8. The needle device of claim 1, wherein the needle has a distal portion bent at an angle relative to a longitudinal axis of a longitudinal portion of the needle.

9. The needle device of claim 8, wherein the angle is at or between 14 degrees and 20 degrees.

10. (canceled)

11. (canceled)

12. The needle device of claim 9, wherein the distal end of the needle is beveled.

13. (canceled)

14. (canceled)

15. The needle device of claim 1, forming part of a kit, the kit further comprising:
a second needle device; and
a guidewire.

16. The kit of claim 15, wherein the needle device is configured to reversibly engage a lymph node, and wherein the second needle device is configured to puncture a thoracic duct.

17. The kit of claim 16, further comprising:
a microcatheter configured to cannulate the thoracic duct over the guidewire.

18. The needle device of claim 1, wherein the needle aperture is positioned through a sidewall of the needle proximal to the distal end of the needle.

19. (canceled)

20. The needle device of claim 18, further comprising:
a channel running parallel to the needle, the channel comprising a channel lumen and a channel aperture, wherein the channel aperture and the channel lumen are in fluid communication, the channel lumen disposed within the needle lumen, and the channel aperture is disposed in the needle sidewall proximal of the needle aperture; and
a securing means disposed in the channel lumen.

21. The needle device of claim 20, wherein the distal end comprises a curved distal tip.

22. (canceled)

23. (canceled)

24. (canceled)

25. (canceled)

26. A needle device, comprising:
a needle having a needle lumen defined therethrough terminating at a needle aperture at or near a distal end of the needle; and
a securing device positioned within the needle lumen, the spiraling element configured to at least partially protrude from the needle aperture and to reversibly engage a tissue;
the needle device configured to reversibly puncture a tissue and further configured to introduce a fluid through the needle lumen into the tissue or a lumen thereof.

27. The needle device of claim 26, wherein the securing device comprises a spiraling element, and wherein the spiraling element is configured to reversibly engage a lymph node.

28. (canceled)

29. (canceled)

30. (canceled)

31. The needle device of claim 27, wherein the spiraling element comprises a helical distal end terminating with a sharp and straight distal tip.

32. The needle device of claim 26, wherein the needle has a distal portion bent at an angle relative to a longitudinal axis of a longitudinal portion of the needle.

33. (canceled)

34. (canceled)

35. (canceled)

36. (canceled)

37. (canceled)

38. (canceled)

39. The needle device of claim 26, forming part of a kit, the kit further comprising:
a second needle device; and
a guidewire.

40. (canceled)

41. The kit of claim 39, further comprising:
a microcatheter configured to cannulate a thoracic duct over the guidewire.

42.-61. (canceled)
